(19) United States
(12) Patent Application Publication
(10) Pub. No.: US 2023/0381591 A1
(43) Pub. Date: Nov. 30, 2023

(54) APPARATUS AND METHOD FOR PROVIDING CUSTOMIZED EXERCISE INFORMATION ON BASIS OF MOTION TRACKING INFORMATION ABOUT USER

(71) Applicant: NUVI LABS CO., LTD., Seoul (KR)

(72) Inventor: Dae Hoon KIM, Gunpo-si (KR)

(73) Assignee: NUVI LABS CO., LTD., Seoul (KR)

(21) Appl. No.: 18/030,925

(22) PCT Filed: Oct. 7, 2021
(86) PCT No.: PCT/KR2021/013825

§ 371 Date: Apr. 7, 2023

(30) Foreign Application Priority Data
Oct. 8, 2020 (KR) 10-2020-0129929

(51) Int. Cl. A63B 24/00; G16H 20/30; G06T 7/292; G06V 40/10; A63B 71/06

(52) U.S. Cl. CPC A63B 24/0075; G16H 20/30; G06T 7/292; G06V 40/15; A63B 71/0622; A63B 24/0062; A63B 24/0003; G16H 50/70

(57) ABSTRACT

According to one example embodiment, an apparatus includes a kiosk device, a plurality of cameras, and a processor, which check an authenticity and an identification information about a specified user by checking the identification information about the user recognized in the image obtained by capturing the space, and then tracks the motion of the user for which the authenticity has been checked using a user tracking algorithm, generate exercise habit information about the user by using bio-signals and motion signals of the user, which are acquired from the time when an exercise using fitness equipment provided in the space starts to the time when the exercise ends, generate exercise method information to be recommended to the user by using the identification information about the user and the exercise habit information about the user, and provides the generated exercise method information to the user.

### TECHNICAL FIELD

According to one embodiment, there are provided an apparatus and method for diagnosing a physical condition of a user based on information tracking motion of the user and biometric information of the user, and providing an exercise method most suitable to the user and various information related to exercise.

### BACKGROUND ART

In recent years, more and more people use the fitness center and home training to improve their strength or health. In particular, weight training using barbell, which requires a very sophisticated motion as a number of joints and muscles are used to improve the muscle strength, is gaining popularity.

However, the weight training motion using fitness equipment provided in a gym may cause injury to muscles or joints and body imbalance unless the proper weight training motion is performed with an appropriate exercising posture.

Thus, it is important to learn the correct weight training motion with the help of experts through personal training. However, the majority of people are reluctant to personal training because of high cost, location or time constraints and professional level deviations. For this reason, the importance of controlling precise motions when performing weight training is steadily pointed out, but it is not practiced properly.

Accordingly, researches on smart exercise assisting devices and systems for personal exercise are actively progressing. However, there are also various technical limitations such as high cost burden, inaccuracy caused by a recognition error or an error caused by clothes, or limited recognition only to the front.

### SUMMARY

An embodiment of the present disclosure relates to an apparatus and method for providing customized exercise information based on motion tracking information about a user, and may be used for user health management in an untact era by determining a current biological state of the user and providing a healthcare service through a virtual reality device.

According to one embodiment, a user may be tracked using a plurality of cameras installed in a space, and the identity of the user may be identified using an identifier of the user, so as to be used for management of the user using the space.

In addition, since a user may analyze exercise habit using a plurality of cameras, a wearable device of the user, a fitness equipment sensor, and the like, it is possible to help the user exercise more accurately and effectively.

A technical task of the present disclosure is to provide an apparatus and method for providing customized exercise information including an exercise method most suitable to a user and a diet by analyzing a body type, constitution, and habit of the user based on motion tracking information about the user.

According to a first example embodiment, there may be provided with an apparatus for providing customized exercise information based on motion tracking information about a user, wherein the apparatus includes: a kiosk device for recognizing identification information about a user having entered a space; a plurality of cameras for tracking motion of the user in the space; and a processor, which check an authenticity and an identification information about a specified user, by checking an identification information about a user recognized in an image obtained by capturing the space, and then tracks the motion of the user for which the authenticity has been checked using a user tracking algorithm, generates exercise record information about the user by using bio-signals and motion signals of the user, the bio-signals and motion signals of the user being acquired between the time when an exercise using a fitness equipment installed in the space starts and the time when the exercise using the fitness equipment ends, generate information to be recommended to the user by using the identification information about the user and the exercise record information about the user, and provides the generated information to the user.

According to a second example embodiment, there may be provided with the apparatus, wherein a first user is verified by acquiring a plurality of identifiers from age, gender, and appearance information of a plurality of users, learning a correlation between the plurality of identifiers and user identification accuracy to learn an artificial intelligence model, and detecting at least one other identifier of a first user using a first identifier extracted from the first user using the artificial intelligence model.

According to a third embodiment, the motion of the user exercising using a first fitness equipment among a plurality of pieces of fitness equipment installed in the space is tracked when force of the user applied to the first fitness equipment is sensed, an exercise state of the user is analyzed through the motion of the user acquired by tracking, exercise coaching information including an exercise posture, an exercise method, and an exercise amount for the first fitness equipment is generated based on the exercise state of the user, and physical condition state information about the user before a start of the exercise is acquired from the user to generate a first emoji indicating a target state of the user and a second emoji of the user indicating a current exercise state of the user.

According to a fourth example embodiment, there may be provided with the apparatus for providing customized exercise information based on motion tracking information about a user, wherein the apparatus is configured to: generate fitness equipment learning information including changes in exercise posture, changes in muscle mass, and changes in body fat while using fitness equipment used by the user based on exercise habit information about the user acquired for a predetermined period; generate a trainer emoji that corrects the exercise posture of the user based on the fitness equipment learning information; generate an exercise similar group according to the identification information of registered users; decide a ranking in the group of the exercise habit information about the user based on the exercise habit information of other users included in the group to which the user belongs; and generate an emoji of the other users based on the exercise habit information of the other users who is one ranking higher than the decided ranking of the user and provide the user therewith.

According to a fifth example embodiment, there may be provided with the apparatus for providing customized exercise information based on motion tracking information about a user, wherein the apparatus is configured to: when the identification information of a second user recognized in the image obtained by capturing the space has not been checked, generate a profile of the second user; decide at least one identifier from appearance information of the second user; and recognize an identity of the second user by mapping and storing the decided at least one identifier and the image of the second user.

According to a sixth example embodiment, there may be provided with a computer program product including a computer-readable storage medium capable of executing a method for providing customized exercise information based on motion tracking information about a user according to one embodiment.

According to a seventh example embodiment, there may be provided with a computer-readable recording medium in which a program for implementing a method for providing customized exercise information based on motion tracking information about a user according to one embodiment is recorded.

### Advantageous Effects

According to one example embodiment, it is possible to determine a current biological state of a user using a wearable device and provide a healthcare service through a virtual reality device, and thus it is effective for user health management in an untact era.

According to one example embodiment, a user may be tracked using a plurality of cameras installed in a space, and the identity of the user may be identified using an identifier of the user, so as to be useful for management of the user using the space.

In addition, since a user may analyze exercise habit using a plurality of cameras, a wearable device of the user, a fitness equipment sensor, and the like, it is possible to help the user exercise more accurately and effectively.

### BRIEF DESCRIPTION OF THE DRAWINGS

FIG. 1 is a diagram for illustrating a technical characteristic of providing customized exercise information based on motion tracking information about a user according to one example embodiment.

FIG. 2 is a flowchart for illustrating a method for providing customized exercise information based on motion tracking information about a user according to one example embodiment.

FIG. 3 is a diagram for illustrating the characteristics of learning and using an artificial intelligence model for accurately identifying a user from an image of a space according to one example embodiment.

FIG. 4 is a diagram for illustrating the characteristics of analyzing an exercise habit of a user according to one example embodiment and providing a user-customized exercise method.

FIG. 5 is a diagram for illustrating the characteristics of a screen that provides information for reaching a target of a user according to one example embodiment.

FIG. 6 is a diagram for illustrating the configuration of an apparatus for providing customized exercise information based on motion tracking information about a user according to one example embodiment.

### DESCRIPTION OF EXEMPLARY EMBODIMENTS

Hereinafter, the present disclosure will be described in detail with reference to the accompanying drawings.

Terms used herein will be briefly described, and disclosed embodiments will be described in detail below.

The terms used herein have been selected as general terms which are widely used at present in consideration of the functions of the present disclosure, and this may be altered according to the intent of an operator skilled in the art, conventional practice, or introduction of new technology. In addition, in a specific case, a term is arbitrarily selected by the applicant, and a meaning of the term will be described in detail in a corresponding description portion of the exemplary embodiments. Thus, the terms used herein should be defined based on the overall content of the present disclosure instead of a simple name of each of the terms.

In this specification, the expressions “have”, “may have”, “include” and “comprise”, or “may include” and “may comprise” used herein indicate existence of corresponding features (e.g., elements such as numeric values, functions, operations, or components) but do not exclude presence of additional features.

The expression “at least one of A and/or B” should be interpreted to mean any one of “A” or “B” or “A and B.”

The expressions “first,” “second” and the like used in this specification may be used to describe various elements regardless of any order and/or degree of importance. In addition, such expressions are used only to distinguish one element from another element, and are not intended to limit the elements.

The description in the disclosure that one element (e.g., a first element) is “(operatively or communicatively) coupled with/to” or “connected to” another element (e.g., a second element) should be interpreted to include both the case where the one element is directly coupled to the another element, and the case where the one element is coupled to the another element through still another element (e.g., a third element).

Singular expressions may include plural expressions, unless defined obviously differently in the context. In addition, in this specification, the terms such as “include” or “consist of” should be construed as designating that there are such characteristics, numbers, operations, elements, components or a combination thereof in the specification, but not to exclude the existence or possibility of adding one or more of other characteristics, numbers, operations, elements, components or a combination thereof.

The terms “a module” or “a part” used in the disclosure are for referring to elements performing at least one function or operation, and these elements may be implemented as hardware or software, or as a combination of hardware and software. Further, a plurality of “modules” or “parts” may be integrated into at least one module and implemented as at least one processor (not shown), except “modules” or “parts” that need to be implemented as specific hardware.

Hereinafter, an apparatus 100 for providing customized exercise information based on motion tracking information about a user will be abbreviated as an customized exercise information providing apparatus 100.

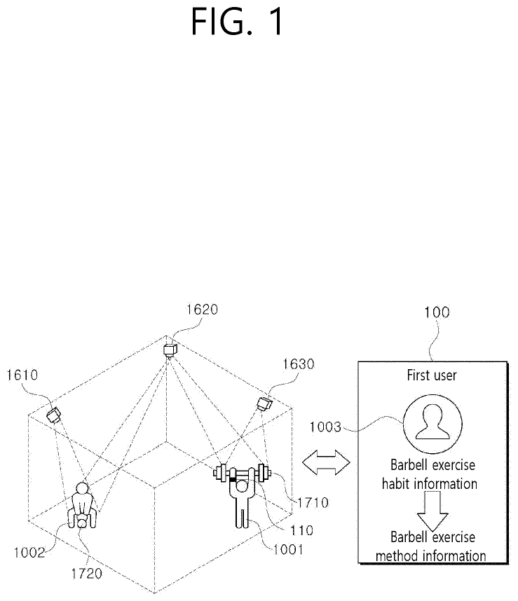

FIG. 1 is a diagram for illustrating a technical characteristic of providing customized exercise information based on motion tracking information about a user according to one example embodiment.

Hereinafter, an apparatus 100 for providing customized exercise information based on motion tracking information about a user will be abbreviated as an customized exercise information providing apparatus 100.

According to one example embodiment, the customized exercise information providing apparatus 100 may acquire identification information of a user entering a space, in particular, a space in which fitness equipment is installed. According to one example embodiment, the customized exercise information providing apparatus 100 may acquire the identification information of the user using a kiosk installed in the space. According to one example embodiment, the customized exercise information providing apparatus 100 may acquire user identification information from the kiosk that recognizes a user terminal, a wearable device of the user, and a recognition device issued to the user.

According to one example embodiment, the customized exercise information providing apparatus 100 may acquire bio-signals of a user from a wearable device 110 that measures the bio-signals of the user. Herein, the bio-signals of the user may include heart rate of the user, blood pressure, body temperature, brain waves, heart rate variability, muscle tone, skin conductivity, respiration rate, blood volume pulse (BVP) and body temperature stress index, body moisture content, and blood sugar. In addition, the wearable device 100 may sense motion signals of the user by sensing the movement of the user.

The wearable device 110 according to one example embodiment may be a smart glove. In addition, the wearable device 100 may be manufactured into smart glasses, smart watches, smart earphones, smart socks, and the like, and may include all smart devices that may be mounted and detached by a user. The user may transmit the biometric information and motion information of the user to the customized exercise information providing apparatus 100 by using the wearable device 110 while exercising in a space.

According to one example embodiment, the customized exercise information providing apparatus 100 may provide a user with content manufactured in augmented reality and virtual reality by using a virtual reality device which provides virtual reality image and audio to the user. The user may experience virtual reality 1001 by using the virtual reality device. The virtual reality device according to one example embodiment may be selectively mounted by the user. The virtual reality device according to one embedment may include any device capable of outputting image and audio for the user to view, such as a VR device, smart glasses, a smart TV, and the like.

According to one example embodiment, the customized exercise information providing apparatus 100 may acquire a plurality of images obtained by capturing a space from a plurality of cameras 1610, 1620, and 1630 installed in a space. The plurality of cameras 1610, 1620, and 1630 may track and capture an object, and may capture the interior of the space while moving within a predetermined movement radius.

The plurality of cameras may acquire 2D or 3D information and provide a user with the recognized identification information about the user, exercise information, and the like.

According to one example embodiment, a plurality of fitness equipment 1710 and 1720 may be provided in a space of an example embodiment of the present disclosure. Each of the plurality of pieces of fitness equipment 1710 and 1720 may be provided with an fitness equipment sensor for measuring a motion of the fitness equipment.

The fitness equipment sensor according to one example embodiment may include a plurality of sensors capable of measuring all motions of the fitness equipment, including movement speed, movement direction, acceleration, inclination, horizontality, and the like of the fitness equipment. The fitness equipment sensor may be provided with a sensor suitable for measuring the motion of each piece of fitness equipment for each type of fitness equipment.

According to one example embodiment, the customized exercise information providing apparatus 100 may recognize a plurality of users 1001 and 1002 in a space. In addition, the customized exercise information providing apparatus 100 may check the identification information about a user recognized in the image obtained by capturing the space, and track the motion of the user using a user tracking algorithm.

According to one example embodiment, the customized exercise information providing apparatus 100 may organically track a specific user through interworking between a plurality of cameras. According to one example embodiment, the customized exercise information providing apparatus 100 may identify a user by using specific appearance information about the user, and may check user information by matching the same with pre-stored information about the user.

According to one disclosure, the customized exercise information providing apparatus 100 may generate exercise record information about a user by using bio-signals or motion signals of the user acquired from a start of an exercise to a completion of the exercise using a plurality of pieces of fitness equipment.

According to one example embodiment, the customized exercise information providing apparatus 100 may generate an emoji 1003 of a user, show an exercise record of the user through a change of the emoji, or show a correct exercise method.

Exercise habit of a user according to one example embodiment may be information generated to determine whether the user 1001 uses the fitness equipment correctly while using specific fitness equipment 1710 and whether exercise efficiency is high.

According to one example embodiment, the customized exercise information providing apparatus 100 may generate exercise method information to be recommended to a user by using the identification information about the user or the exercise record information about the user. In other words, it is possible to provide the user with customized information that may help the user exercise based on a physical condition of the user and a final target desired by the user.

The customized information provided to the user means information such as an exercise method, an exercise target, a diet control, a customized trainer, and a nutritional supplement in consideration of an exercise state of a user, and encompasses healthcare information that may be provided to a user and is not limited to the preceding example.

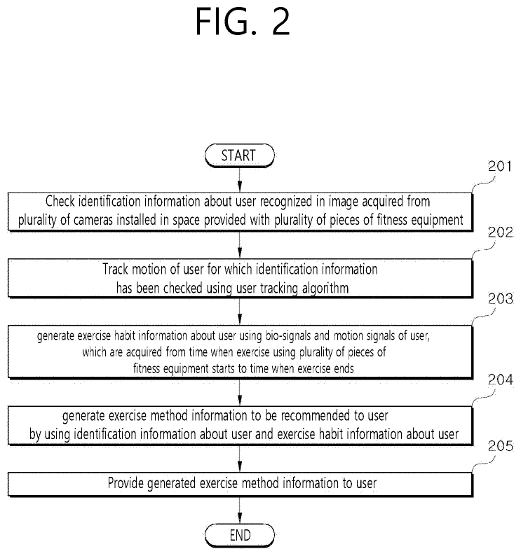

FIG. 2 is a flowchart for illustrating a method for providing customized exercise information based on motion tracking information about a user according to one example embodiment.

In block 201, according to one example embodiment, the customized exercise information providing apparatus 100 may provide checking identification information about a user recognized in an image acquired from a plurality of cameras installed in a space having a plurality of pieces of fitness equipment.

In block 202, according to one example embodiment, the customized exercise information providing apparatus 100 may provide tracking a motion of a user for which the identification information has been checked using a user tracking algorithm.

In this connection, the customized exercise information providing apparatus 100 may track the motion of the user and use the analyzed information and/or use the information of an electronic tag mounted by the user to understand a route of the user.

A user according to one example embodiment may mount a module including an identification marker including a UVB tag or an RFID tag. For example, it is possible to mount an athletic uniform inducing the identification marker.

The identification marker is a module including a UWB tag or an RFID tag manufactured to be mounted and detached by the user. The identification marker may be included in the uniform or may be manufactured in the form of an armband that may be mounted and detached, or an accessory that may be carried, and the making method is not limited.

A Radio Frequency Identification (RFID) technology may recognize an object by identifying unique information using radio waves, and may perform location tracking accordingly. The RFID technology is a technology that integrates the use of electromagnetic or electrostatic coupling within the radio frequency of an electromagnetic spectrum to identify objects, animals, or humans. The biggest advantage of RFID is that there is no need for direct contact or scanning in a visible band. An RFID system includes an antenna, a reader, and a tag, also called a transponder. The antenna uses radio frequency waves to carry a signal to activate the tag. When the tag is activated, the data of the tag is transmitted to the antenna. The antenna is connected to the reader to receive data from the tag, so that data transmission using RFID is performed. Herein, tags are divided into active and passive types. Active tags may transmit data even in a range exceeding a distance of several tens of meters from the reader, and passive tags may transmit data only at a close distance of several centimeters.

The UWB tag may continuously transmit a UWB impulse signal for location tracking using an ultra-wideband (UWB) communication technology. The UWB tag is a tag provided to each user, that is, a monitoring object, and may be an access card type, a badge type, or the like. An ID for identifying each tag may be assigned to the UWB tag, and by assigning a monitoring priority, tracking and monitoring may be performed according to the priority assigned to the UWB tag when several monitoring objects compete. UWB-based location tracking uses a UWB wireless communication technology that uses a very wide frequency band from 500 MHz to several GHz or more, and has recently been developed for the purpose of precisely tracking a location in a narrow space, such as indoors, without GPS. The UWB tag may generate an impulse by encoding data, and may transmit a UWB impulse signal through a UWB transmission antenna.

The RFID tag and the UWB tag are not heavy in weight and do not have a large volume, so they have the advantage of portability, so they are easy to be manufactured as a uniform, additional accessory, or armband of a user.

The customized exercise information providing apparatus 100 according to one example embodiment may receive a signal from the identification marker and analyze the signal from the UWB tag and the RFID tag to secure a movement route of a user, or analyze a camera image to track and analyze the identification marker to secure the movement route of the user.

In block 203, according to one example embodiment, the customized exercise information providing apparatus 100 may provide generate exercise record information about a user by using bio-signals or motion signals of the user, which are acquired from the time when an exercise using a plurality of pieces of fitness equipment starts to the time when the exercise ends.

The exercise record information acquired in the block 203 may generate exercise record information of a user through information provided from images acquired from a plurality of cameras, and optionally, each of the plurality of pieces of fitness equipment may include a motion sensor capable of measuring a motion of fitness equipment. The sensor includes all sensors that may be mounted on the fitness equipment by those skilled in the art, and a detailed description will be omitted.

In block 204, according to one example embodiment, the customized exercise information providing apparatus 100 may provide generating exercise method information to be recommended to a user by using the identification information about the user or the exercise record information about the user.

In block 205, according to one example embodiment, the customized exercise information providing apparatus 100 may provide the generated exercise method information to a user.

According to one example embodiment, the customized exercise information providing apparatus 100 may detect a first identifier indicating a characteristic of a first user recognized from a space-captured image. The first identifier indicates the external characteristics that may be identified through a camera image, and may indicate appearance information about a user including, for example, height, head, shoulder width, hair color, and the position of the crown.

According to one example embodiment, when there is a first user profile matching the first identifier, the customized exercise information providing apparatus 100 may detect a second identifier included in the first user profile from an image. Herein, the user profile may include presently verified information about a user, and may include information about an identifier capable of authenticating each user. The user profile according to one example embodiment may include an identifier for at least one user.

According to one example embodiment, when the second identifier is detected, the customized exercise information providing apparatus 100 may determine the reliability of detection of a first user to be greater than or equal to a threshold value and determine the first user as the tracking object. The second identifier is an identifier indicating a characteristic of a user other than the presently detected first identifier.

According to one example embodiment, the customized exercise information providing apparatus 100 may include information on a plurality of identifiers determined in advance for the first user, and as the number of identifiers matching the identifier of a user increases in the space-captured image, the reliability of first user detection may be highly evaluated. The high reliability of the first user detection means that the user detected in the image is a previously verified specific user.

According to one example embodiment, the customized exercise information providing apparatus 100 may continuously track a motion of a user determined as a tracking object in a space through analysis of a plurality of camera images. The tracking object according to one example embodiment is a moving person, and the customized exercise information providing apparatus 100 may track a route of the person determined as a tracking object through moving of a plurality of cameras or analysis of a plurality of camera images.

According to one example embodiment, when at least two or more identifiers are detected, the customized exercise information providing apparatus 100 may finalize a user as a specific person. In this connection, it is determined that the identification information about the user has been verified.

According to one example embodiment, the customized exercise information providing apparatus 100 may track a motion of an object in which the first identifier and the second identifier are simultaneously detected. In addition, the customized exercise information providing apparatus 100 may generate a route of the tracked object as time-series information and store the same in the database.

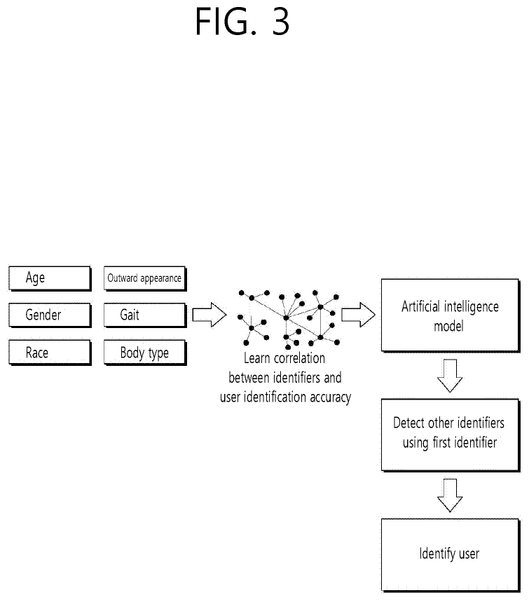

FIG. 3 is a diagram for illustrating the characteristics of learning and using an artificial intelligence model for accurately identifying a user from an image of a space according to one example embodiment.

According to one example embodiment, the customized exercise information providing apparatus 100 may acquire a plurality of identifiers from age, gender, and appearance information of a plurality of users, and learn the correlation between the plurality of identifiers and user identification accuracy to learn an artificial intelligence model.

Herein, the appearance information includes the outward appearance of facial features, crown position, head shape, hair style, etc. of a person, and furthermore, may include body type information such as a gait, posture, exercise posture, and shoulder height of a person. The appearance information may include all characteristics that are identification values that distinguish people, but is not limited thereto.

According to one example embodiment, the customized exercise information providing apparatus 100 may generate an artificial intelligence model by learning the correlation between the identifier and the user identification accuracy by learning the characteristic data of thousands or tens of thousands of people.

According to one example embodiment, the customized exercise information providing apparatus 100 uses an artificial intelligence model to verify the first user by detecting at least one other identifier of the first user using the first identifier extracted from the first user.

According to one example embodiment, the customized exercise information providing apparatus 100 may identify a user using an identifier of a specific person detected from an image using the generated artificial intelligence model. According to one example embodiment, the customized exercise information providing apparatus 100 may check identification information of a specific person by detecting an identifier other than the first identifier visited for the first time.

According to one example embodiment, the customized exercise information providing apparatus 100 may generate a profile of the second user when the identification information of the second user recognized in the image obtained by capturing the space has not been checked.

According to one example embodiment, the customized exercise information providing apparatus 100 may decide at least one identifier from appearance information of the second user, and map and store the decided at least one identifier and the image of the second user to generate a new profile.

According to one example embodiment, the customized exercise information providing apparatus 100 may transmit a notification message for the generation of a new profile of the second user to a manager to request the manager to check a new user.

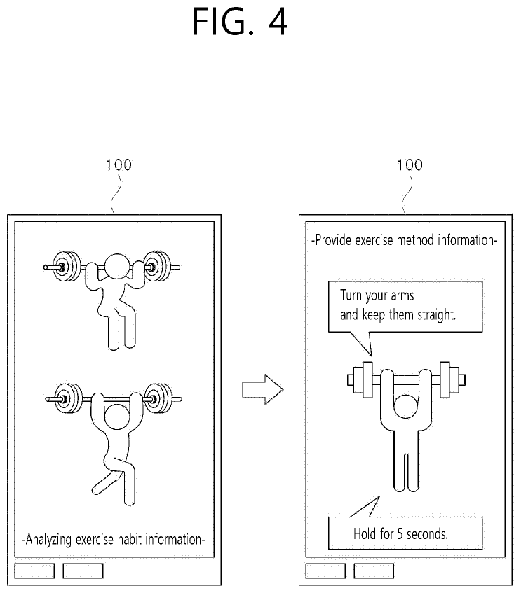

FIG. 4 is a diagram for illustrating the characteristics of analyzing an exercise habit of a user according to one example embodiment and providing a user-customized exercise method.

According to one example embodiment, the customized exercise information providing apparatus 100 may track a motion of a user exercising by using the first fitness equipment when detecting the user force applied to the first fitness equipment among the plurality of pieces of fitness equipment.

According to one example embodiment, the customized exercise information providing apparatus 100 may acquire information about an exercise state of a user by capturing the movement of the first fitness equipment by a plurality of cameras, optionally acquires angular velocity data and acceleration data of the first fitness equipment while the user is exercising through the fitness equipment sensor included in the first fitness equipment, and analyze the exercise state of the user by analyzing the angular velocity data and the acceleration data of the first fitness equipment.

According to one disclosure, the customized exercise information providing apparatus 100 may generate exercise coaching information including an exercise posture, an exercise method, and an exercise amount for the first fitness equipment based on an exercise state of a user, and provide the user therewith.

According to one example embodiment, the customized exercise information providing apparatus 100 may provide exercise method information to a user using a user terminal or a virtual reality device, or may provide customized exercise method information through a display installed in a space.

According to one example embodiment, the customized exercise information providing apparatus 100 may help a user exercise by providing a customized exercise method through a mirror display.

According to one example embodiment, the customized exercise information providing apparatus 100 may generate fitness equipment learning information including changes in exercise posture, changes in muscle mass, and changes in body fat while using fitness equipment used by a user based on the exercise record information about the user acquired for a predetermined period.

According to one example embodiment, the customized exercise information providing apparatus 100 may generate a trainer emoji that corrects an exercise posture of a user based on the fitness equipment learning information. According to one example embodiment, the customized exercise information providing apparatus 100 generates an emoji of a trainer and shows the shape of directly executing an exercise method, thereby effectively helping a user exercising using specific fitness equipment.

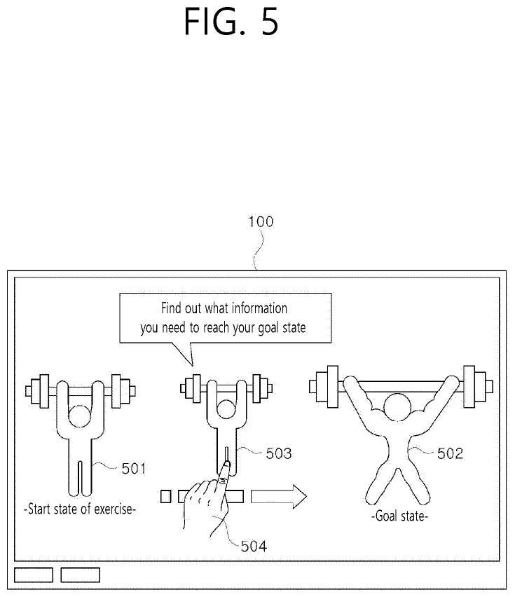

FIG. 5 is a diagram for illustrating the characteristics of a screen that provides information for reaching a target of a user according to one example embodiment.

According to one example embodiment, the customized exercise information providing apparatus 100 may acquire physical condition information about a user before a start of an exercise from the user, and may generate a first emoji 501 of the user indicating an initial state of the user. In other words, the first emoji 501 is a character generated by reflecting a physical condition of the user at the time of starting the exercise using the relevant fitness equipment.

According to one example embodiment, the customized exercise information providing apparatus 100 may acquire a final exercise target of a user from the user and generate a second emoji 502 of the user indicating a target state of the user. Herein, the customized exercise information providing apparatus 100 may include a target body shape of the user, such as the amount of body fat and muscle strength desired by the user, and may include an exercise target such as the weight of the relevant fitness equipment and the posture of the user.

According to one example embodiment, the customized exercise information providing apparatus 100 may generate a third emoji 503 of a user indicating a current state of the user reflecting the change from an initial state of the user by using the bio-signals of the user and the exercise habit information about the user acquired after the user started exercising. In other words, the third emoji 503 represents a user state at a current time point.

The first emoji 501, the second emoji 502, and the third emoji 503 are individually provided to a user, and are provided as information on an initial state of the user, a state for the final target, and a current state.

According to one example embodiment, the customized exercise information providing apparatus 100 may compare specifications of the third emoji 503 with specifications of the first emoji 501 and the second emoji 502. In other words, by simultaneously displaying an initial state of a user, a final target state of the user, and a current state of the user, it is possible to visually display where the current state of the user is located.

Herein, the specification of the emoji may include information such as the user's biometric information, muscle strength information, and posture information at the time of generating the emoji or the user's target time.

According to one example embodiment, the customized exercise information providing apparatus 100 may display the third emoji 503 between the first emoji 501 and the second emoji 502. When it is determined that a physical condition of a user is lower than the specification of the first emoji 501, the third emoji 503 may be displayed before the first emoji 501.

According to one example embodiment, the customized exercise information providing apparatus 100 may display the specifications of the third emoji 503 for each item in response to receiving an input 504 of a user selecting the third emoji 503.

According to one example embodiment, the customized exercise information providing apparatus 100 may generate an exercise similar group according to the identification information of registered users. A standard of dividing groups vary, such as age, gender, region of residence, type of exercise, and the like, and similar groups may be generated for each of a plurality of standards. In addition, a standard of dividing a group may be decided by a user's selection.

According to one example embodiment, the customized exercise information providing apparatus 100 may decide a ranking in the group of the exercise record information about the user based on the exercise habit information of other users included in the group to which the user belongs. According to one example embodiment, the customized exercise information providing apparatus 100 may generate an emoji of the other users based on the exercise record information of the other users who are one ranking higher than the decided user and provide the user therewith.

According to one example embodiment, the customized exercise information providing apparatus 100 may decide a ranking of each user based on how much a user approaches a target value using information of users in a similar group. Herein, the criterion for deciding the ranking is made based on whether a target value is reached, in other words, the user closest to the target set by the user has the highest priority.

According to one example embodiment, the customized exercise information providing apparatus 100 provides a reward to a user based on whether each user has reached a target value. For example, when the first user has a higher ranking than other users in the similar group, a higher reward than other users may be provided.

For example, the processor may calculate the reward R to be provided to a user based on Equation 1 below based on the scores Su of other users decided depending on how much the target set value has been reached.

R−\sqrt{(1−Sᵤ)²} [Equation 1]

According to Equation 1, the reward R to be provided to the user may increase as the scores of other users increase.

The processor may generate an emoji of a user, generate a first motion vector from a first motion of a user, and generate a second motion vector by multiplying a weighting according to the first motion vector and at least one biometric information value by the first motion vector according to Equation 2 below.

F_{N}=a₁×T₁+a₂×T₂+a₃/T₃ [Equation 2]

Herein, FN is a second motion vector, a1, a2, and a3 denote weightings previously generated according to biometric information values, and T1, T2, and T3 denote vector values corresponding to the weightings. For example, a1 may be expressed as 0.2 as a respiration value, a2 as 0.5 as heart rate, and a3 as 0.4 as body temperature, and the weightings may be set differently according to the health condition and personal physical condition information about a user. T1, T2 and T3 denote vectors corresponding to biometric information values. For example, T1 may represent a motion vector of an emoji according to a respiration value.

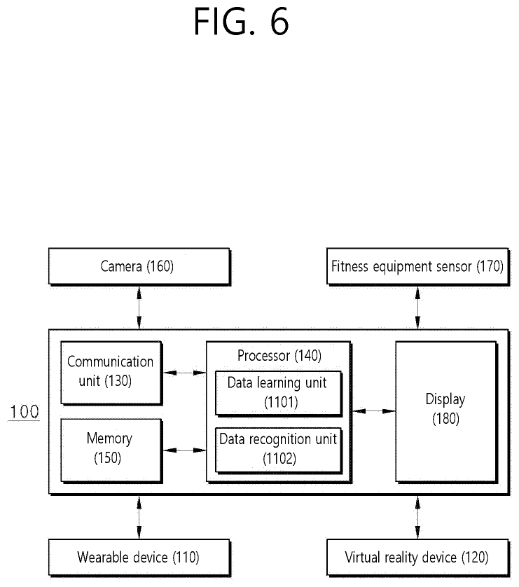

FIG. 6 is a diagram for illustrating the configuration of an apparatus for providing customized exercise information based on motion tracking information about a user according to one example embodiment.

According to one example embodiment, the customized exercise information providing apparatus 100 may be configured as a communication unit 130, a memory 150, a processor 140, and a display 180, and may include a wearable device 110, a virtual reality device 120, a camera 160, and a fitness equipment sensor 170 as a configuration.

The camera 160 according to one example embodiment may include a plurality of camera modules installed in a space. The plurality of camera modules may be manufactured as a movable or rotatable module, and may capture an image of a space and transmit the same to the processor 140.

The display 180 according to one example embodiment is a device for providing customized exercise information, and may be a display that is included as a component of the customized exercise information providing apparatus 100 or is located outside, and may be a module enabling the communication with the processor 140 through the communication unit 130.

The wearable device 110 according to one example embodiment may include a smart glove mountable on a hand of a user. Furthermore, the wearable device 110 may include a device mountable by a user including smart glasses, a smart watch, smart shoes, and the like.

The wearable device 110 may include a plurality of sensors for sensing user bio-signals including a motion of a user, blood pressure, body temperature, brain waves, heart rate, heart rate variability, muscle tone, skin conductivity, respiration rate, blood volume pulse (BVP) and body temperature stress index, body moisture content, and blood sugar. A detailed description of the plurality of sensors will be omitted.

According to one example embodiment, the wearable device 110 may include an antenna, a signal reception unit for receiving a radio signal having a frequency of 860-930 MHz from a QR code through the antenna, and a signal control unit that is connected to the signal reception unit and analyzes information included in the received QR code.

According to one example embodiment, the antenna may include a coating layer manufactured by including 30 to 40 parts by weight of an insulator based on 100 parts by weight of an epoxy acrylate resin represented by Formula 1 below.

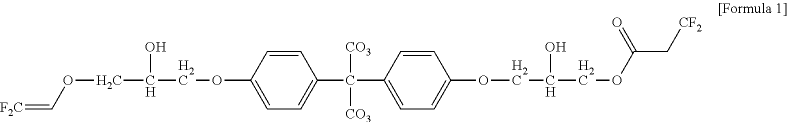

An insulation coating composition includes an epoxy acrylate resin and an insulator, and the insulator may specifically be formed of oxides such as silicon dioxide, Al2O3, Ta2O5, TiO2, CrO2, HfO2, Y2O3, PMMA, and TEOS, nitrides such as silicon nitride, tantalum nitride, and titanium nitride, oxynitrides such as SiOxNy, high dielectric constant (ε>30) metal oxides such as BaSiTiO3, BaTiO3, and SrTiO3, xerogels and combinations, multilayers, or mixtures thereof.

In addition, the coating layer may be formed on a substrate using a deposition technique. For example, chemical vapor deposition, physical vapor deposition, sputtering, vacuum deposition, spin-on coating, dip coating, and other similar deposition techniques may be used to form the coating layer.

### Preparation Example

An insulation coating composition was prepared by mixing 35 parts by weight of an insulator, 5 parts by weight of a curing agent monoamine, and 100 parts by weight of a solvent ethyl acetate, based on 100 parts by weight of the epoxy acrylate resin represented by Formula 1 above. As the insulator, Ta2O5, TiO2, CrO2, and Y2O3 were mixed and used in the same ratio.

The insulation coating composition was coated on one surface of the substrate to a thickness of 2 nm to form a coating layer.

### Comparative Example 1

The insulation coating composition was prepared in the same manner as in the Preparation Example except that the epoxy acrylate resin was excluded.

### Comparative Example 2

The insulation coating composition was prepared in the same manner as in Preparation Example except that the insulator was excluded.

### Experimental Example 1

### Insulation Property Evaluation

The surface resistance of the substrate was measured using a high resistance tester of Mitsubishi, Japan.

TABLE 1

| | Preparation | Comparative | Comparative |
|---|---|---|---|
| | Example | Example 1 | Example 2 |
| Surface resistance | ○ | Δ | x |

(○: surface resistance value ranging more than 3 × 1010,
Δ: surface resistance value ranging from 1 × 109 to 3 × 1010,
x: surface resistance value ranging less than 1 × 109)

According to the experimental results, it was identified that the insulating effect was excellent when the insulation coating layer of an example embodiment of the present disclosure was formed.

### Experimental Example 2

### Insulation Film Reliability Evaluation

In order to evaluate the peel-off characteristics, PCT evaluation was performed using a PCT chamber of Hirayama, Japan.

The aforementioned PCT evaluation was conducted under conditions of a temperature of 121° C., a pressure of 2 atm, and a relative humidity (RH) of 100%.

In addition, in order to evaluate the peel-off characteristics, constant temperature and humidity evaluation was performed using COSMOPIA of Hitachi, Japan. The aforementioned constant temperature and humidity evaluation was conducted under the conditions of a temperature of 85° C. and a relative humidity of 85%.

TABLE 2

| | Preparation | Comparative | Comparative |
|---|---|---|---|
| | Example | Example 1 | Example 2 |
| peel-off (PCT Test) | x | x | ○ |
| peel-off (constant temperature | x | ○ | ○ |
| and humidity test) | | | |

(○: The coating layer is detached from the substrate.
x: The coating layer is not detached from the substrate) In order to check whether the coating layer is detached under the conditions of use of the substrate, a peel-off test was performed.

As for peel-off, it was identified that the coating layer according to the preparation example of the present disclosure was not detached. Also in the case of the comparative examples, it was identified in Comparative Example 1 that a detachment was not found in a PCT test, whereas a detachment was found in the constant temperature and humidity test.

The virtual reality device 120 combines the virtual word having additional information with the real world in real time and shows the same as one image, and thus may include a device configured to be viewable by being interlinked with a smartphone that shows Mixed Reality (MR) or by mixing virtual images with real images through data received through other communication networks.

The virtual reality device 120 may include smart glasses, smart goggles, and the like. For example, by displaying a composite virtual reality environment in which a real image environment collected by a user wearing smart glasses and a virtual area environment based on augmented reality are fused on a field of view of the user, a corresponding target image or position image a user wants to acquire is converted into a three-dimensional composite image, so as to be more efficiently transmitted to the user.

The virtual reality device 120 according to one example embodiment may include a plurality of sensor modules capable of measuring bio-signals of a user, such as brain waves, motion, respiration, and body temperature of the user.

According to one example embodiment, the customized exercise information providing apparatus 100 may include a memory 150 for storing one or more instructions and a processor 140 for executing the one or more instructions stored in the memory 150. According to one example embodiment, the processor 140 included in the customized exercise information providing apparatus 100 may perform the method of an example embodiment of the present disclosure by executing the one or more instructions.

The processor 140 generally controls the overall operation of the customized exercise information providing apparatus 100. For example, the processor 140 may control the other overall configurations included in the customized exercise information providing apparatus 100 by executing programs stored in the memory 150. In addition, the processor 140 may execute the programs stored in the memory 150 to perform the function of the customized exercise information providing apparatus 100. The processor 140 may include at least one processor. The processor 140 may include a plurality of processors or a single processor in an integrated form according to functions and roles thereof. In an example embodiment, the processor 140 may include at least one processor for providing a notification message by executing at least one program stored in the memory 150.

The processor 140 according to an example embodiment may include a data learning unit 1101 and a data recognition unit 1102.

At least one of the data learning unit 1101 and the data recognition unit 1102 may be manufactured as at least one hardware chip and then mounted to an electronic device. For example, at least one of the data learning unit 1101 and the data recognition unit 1102 may be manufactured in the form of a dedicated hardware chip for artificial intelligence (AI) or manufactured as a part of a conventional general purpose processor (for example, CPU or application processor) or a dedicated graphics processor (for example, GPU) and then mounted to the above mentioned electronic device.

In this connection, the data learning unit 1101 and the data recognition unit 1102 may be mounted on one electronic device or on separate electronic devices, respectively. For example, one of the data learning unit 1101 and the data recognition unit 1102 may be contained in an electronic device and the other may be contained in a server. Through the wired or wireless communication, the data learning unit 1101 and the data recognition unit 1102 may provide the data recognition unit 1102 with the model information constructed by the data learning unit 1101, and may provide the data learning unit 1101 with additional training data, which is input to the data recognition unit 1102.

At least one of the data learning unit 1101 and the data recognition unit 1102 may be implemented as a software module. When at least one of the data learning unit 1101 and the data recognition unit 1102 is implemented as a software module (or a program module including instructions), the software module may be stored in non-transitory computer readable media. In addition, in this connection, at least one software module may be provided by Operating System (OS) or a certain application. Alternatively, some of the at least one software module may be provided by the OS and the remaining some may be provided by a certain application.

The memory 150 may store a program for processing and control of the processor 140, and may store data input to or output from the customized exercise information providing apparatus 100.

The memory 150 may include a storing medium having at least one type of a flash memory type, a hard disk type, a multimedia card micro type, a card type (for example, a SD memory, a XD memory 150 or the like), a random access memory (RAM) a static random access memory (SRAM), a read-only memory (ROM), an electrically erasable programmable read-only memory (EEPROM), a programmable read-only memory (PROM), a magnetic memory, a magnet disc, or an optical disc.

Programs stored in the memory 150 may be classified into a plurality of modules according to their functions. Herein, the plurality of modules are software, not hardware, and mean a module that operates functionally.

The memory 150 may store a program for processing and control of the processor 140, and may store an image input to the from the customized exercise information providing apparatus 100 or guide information output from the from the customized exercise information providing apparatus 100. In addition, the memory 150 may store specific information for determining whether to output the guide information.

The memory 150 may include a storing medium having at least one type of a flash memory type, a hard disk type, a multimedia card micro type, a card type (for example, a SD memory, a XD memory 150 or the like), a random access memory (RAM) a static random access memory (SRAM), a read-only memory (ROM), an electrically erasable programmable read-only memory (EEPROM), a programmable read-only memory (PROM), a magnetic memory, a magnet disc, or an optical disc.

Programs stored in the memory 150 may be classified into a plurality of modules according to their functions, for example, may be classified into a UI module, a touch screen module, a notification module, and the like.

The UI module may provide a specialized UI, GUI, etc. that is interlocked with the customized exercise information providing apparatus 100 for each application. The touch screen module may sense a touch gesture of a user via the touch screen and transmit information about the touch gesture to the processor 140. The touch screen module according to an example embodiment may recognize and analyze a touch code. The touch screen module may be configured as separate hardware including a controller.

The notification module may generate a signal for notifying the occurrence of an event of the customized exercise information providing apparatus 100. Examples of events occurred by the customized exercise information providing apparatus 100 include call signal reception, message reception, key signal input, schedule notification, and the like.

The processor 140 generally controls the overall operation of the customized exercise information providing apparatus 100. For example, the processor 140 may control the other overall modules by executing programs stored in the memory 150. In addition, the processor 140 may execute the programs stored in the memory 150 to perform the function of the customized exercise information providing apparatus 100.

In addition, the processor 140 may communicate with other devices and other servers using the communication unit 130. The communication unit may include one or more components that allow the customized exercise information providing apparatus 100 to communicate with other devices (not shown) and servers (not shown). The other device (not shown) may be a computing device such as the customized exercise information providing apparatus 100 or a sensing device, but is not limited thereto. For example, the communication unit may include a short-range communication unit, a mobile communication unit, and a broadcast reception unit.

The short-range communication unit may include a Bluetooth communication unit, a Bluetooth low energy (BLE) communication unit, a near field communication unit, a WLAN (Wi-Fi) communication unit, a Zigbee communication unit, an infrared data association (IrDA) communication unit, a Wi-Fi direct (WFD) communication unit, an ultra wideband (UWB) communication unit, and an Ant+ communication unit, but is not limited thereto.

The mobile communication unit transmits and receives a wireless signal to and from at least one of a base station, an external terminal, and a server on a mobile communication network. Herein, the wireless signal may include various types of data associated with transmission/reception of a voice call signal, a video call signal, or a text/multimedia message.

The broadcast reception unit receives a broadcast signal and/or broadcast-related information from the outside through a broadcast channel. The broadcast channel may include a satellite channel and a terrestrial channel. The customized exercise information providing apparatus 100 may not include the broadcast reception unit according to some example embodiments.

An example embodiment may be implemented as a recording medium including computer-readable instructions such as a computer-executable program module. The computer-readable medium may be an arbitrary available medium accessible by a computer, and examples thereof include all volatile and non-volatile media and separable and non-separable media. Further, examples of the computer-readable medium may include a computer storage medium and a communication medium. Examples of the computer storage medium include all volatile and non-volatile media and separable and non-separable media, which are implemented by an arbitrary method or technology, for storing information such as computer-readable instructions, data structures, program modules, or other data. The communication medium typically includes computer-readable instructions, data structures, program modules, other data of a modulated data signal, or other transmission mechanisms, and examples thereof include an arbitrary information transmission medium.

In addition, as used herein, the term “unit” may refer to a hardware component such as a processor or circuit, and/or a software component executed by a hardware component such as a processor.

The above description is merely illustrative of the present disclosure, and one of ordinary skill in the art to which the present disclosure pertains should understand that the present disclosure may be easily modified in other specific forms without changing the technical spirit or essential features of the present disclosure. Accordingly, the example embodiments described above are illustrative in all aspects and should not be understood as limiting. For example, each element described as having an integrated form may be embodied in a distributed manner, and likewise, elements which are described as being distributed may be embodied in an integrated form.

The scope of the present disclosure is defined by the claims below, and all modifications or modified forms derived from the meaning and scope of the claims and concepts equivalent thereto should be interpreted as belonging to the scope of the present disclosure.

1.-6. (canceled)

7. An apparatus for providing customized exercise information based on motion tracking information about a user, the apparatus including:
a kiosk device for recognizing identification information about a user having entered a space;
a plurality of cameras for tracking motion of the user in the space; and
a processor, which check an authenticity and an identification information about a specified user, by checking an identification information about a user recognized in an image obtained by capturing the space, and then tracks the motion of the user for which the authenticity has been checked using a user tracking algorithm, generates exercise record information about the user by using bio-signals and motion signals of the user, the bio-signals and motion signals of the user being acquired between the time when an exercise using a fitness equipment installed in the space starts and the time when the exercise using the fitness equipment ends, generate information to be recommended to the user by using the identification information about the user and the exercise record information about the user, and provides the generated information to the user.

8. The apparatus of claim 7, wherein the processor is configured to:
tracking the motion of the user by using at least one of a movement route of the user for which the authenticity has been checked, an amount of change in biometric information, motion during use of a plurality of pieces of equipment, a healthcare goal set by the user, and exercise habit information of other users;
generate the exercise record information about the user based on the tracked user motion; and
generate and provide customized exercise method information and healthcare information to be provided to the user based on the exercise record information about the user.

9. The apparatus of claim 7, wherein the processor is configured to:
track the motion of the user exercising using a first fitness equipment among a plurality of pieces of fitness equipment installed in the space when force of the user applied to the first fitness equipment is sensed;
analyze an exercise state of the user through the motion of the user acquired by tracking;
generate exercise coaching information including an exercise posture, an exercise method, and an exercise amount for the first fitness equipment based on the exercise state of the user; and
acquire physical condition state information about the user before a start of the exercise from the user to generate a first emoji indicating a target state of the user and a second emoji of the user indicating a current exercise state of the user.

10. The apparatus of claim 7, wherein the processor is configured to:
generate a profile of a second user when an identification information of a second user recognized in the image obtained by capturing the space has not been checked;
decide at least one identifier from appearance information of the second user; and
recognize an identity of the second user by mapping and storing the decided at least one identifier and the image of the second user.

11. The apparatus of claim 7, wherein the processor is configured to generate an exercise similar group based on the exercise record information and provide the user with exercise record information of other user among the exercise similar group.

12. The apparatus of claim 7, wherein the processor is configured to generate the exercise similar group according to the identification information of registered users,
wherein the standard for dividing the exercise similar group includes at least one of age, gender, region of residence, and type of exercise.

12. The apparatus of claim 7, wherein the processor is configured to decide a ranking in the group of the exercise record information about the user based on the exercise habit information of other users included in the group to which the user belongs and generate an emoji of the other users based on the exercise record information of the other users who are one ranking higher than the decided user and provide the user therewith.

13. The apparatus of claim 7, wherein the processor is configured to decide the ranking of each user based on how much the user approaches a target value using information of users in the exercise similar group,
wherein the criterion for deciding the ranking is made based on whether the target value is reached.

14. The apparatus of claim 7, wherein the processor is configured to provide a reward to the user based on whether each user has reached the target value.

15. The apparatus of claim 7, wherein the processor is configured to generate a first image indicating an initial state of the user, a second image indicating a target state of the user, and a third image indicating a current exercise state of the user reflecting the change from the initial state of the user through the acquired physical condition information about the user before a start of an exercise from the user, wherein the first image to the third image are simultaneously displayed on a display, and control to visually display the user's current exercise state by changing the display position of the third image.

16. The apparatus of claim 7, wherein the processor is configured to acquire information about an exercise state of the user by capturing the movement of the first fitness equipment by the plurality of cameras, acquire angular velocity data and acceleration data of the first fitness equipment while the user is exercising through the fitness equipment sensor included in the first fitness equipment, and analyze the exercise state of the user by analyzing the angular velocity data and the acceleration data of the first fitness equipment.

17. A method for providing customized exercise information based on motion tracking information about a user, the method including:
recognizing identification information about a user having entered a space using a kiosk device;
checking an authenticity and an identification information about a specified user by checking an identification information about a user recognized in an image acquired from a plurality of cameras for tracking a motion of the user in the space;
tracking the motion of the user for which the authenticity has been checked using a user tracking algorithm;
generating an exercise record information about the user by using bio-signals and motion signals of the user, the bio-signals and motion signals of the user being acquired between the time when an exercise using a fitness equipment installed in the space starts and the time when the exercise using the fitness equipment ends; and
generating information to be recommended to the user by using the identification information about the user and the exercise record information about the user, and providing the generated information to the user.

18. A computer program stored in a computer-readable recording medium in combination with a computer, which is hardware, to perform the method of claim 17.
